(19) United States
(12) Patent Application Publication
(10) Pub. No.: US 2017/0121723 A1
(43) Pub. Date: May 4, 2017

(54) TRANSFORMATION METHOD OF SUGAR BEET PROTOPLASTS BY TALEN PLATFORM TECHNOLOGY

(71) Applicant: SESVanderHave N.V., Tienen (BE)

(72) Inventors: Guy WEYENS, Beersel (BE); Marc LEFÉBVRE, Jodoigne Souveraine (BE); Shivegowda THAMMANNAGOWDA, Zaventem (BE); Murielle LOMMEL, Itterbeek (BE)

(21) Appl. No.: 15/318,295

(22) PCT Filed: Jun. 12, 2015
(86) PCT No.: PCT/EP2015/063216

§ 371 Date: Dec. 12, 2016

(30) Foreign Application Priority Data
Jun. 12, 2014 (EP) PCT/EP2014/062290

(51) Int. Cl. C12N 15/82

(52) U.S. Cl. CPC C12N 15/8213

(57) ABSTRACT

A method for transformation of sugar beet protoplasts includes obtaining protoplasts from stomatal guard cells isolated from a sugar beet plant. The protoplasts are transformed with a nucleic acid construct including a nucleotide sequence of interest and Transcription Activator-Like Effector Nucleases (TALEN) or one or more vectors including sequences encoding these Transcription Activator-Like Effector Nucleases (TALEN)sequences. The TALEN target and process a target sequence and replace the target sequence through homologous recombination with the nucleic acid construct including the nucleotide sequence of interest, -possibly applying to an in vitroculture of the protoplasts, a medium that is toxic, preferably lethal to the in vitroculture of the protoplasts. Sugar beet plants are regenerated from the cell culture, preferably from the surviving protoplasts having integrated the nucleic acid construct including the sequence of interest that possibly renders the transformed cell resistant to the toxic activity of the applied medium.

### FIELD OF THE INVENTION

The present invention is in the field of plant biotechnology, especially related to a method and means (or tools) for the efficient transformation of sugar beet protoplasts by exogenous sequences.

### BACKGROUND OF THE INVENTION

The ability to modify chromosomes through homologous recombination (gene targeting) is needed for a long time in the field of plant genetics, because gene targeting can target specific sequences or nucleotides resulting into modifications of specific plant functions and opening new possibilities for crops improvement, for increased production of new seeds with required carbohydrate profile or with enhanced nutritional qualities, or for increasing resistance to diseases and stress.

Transcription Activator-Like Effector Nucleases (TALEN) sequences originating from plant pathogen bacteria of the genus xanthomonas, play important role in diseases or trigger defence, by binding host DNA and activating effector-specific host genes. TALEN's are specific transcription activator-like effector nucleases (artificial restriction enzymes) that can be engineered to bind and cut the specific DNA sequences in the genome. Once these TALEN's are introduced into cells, they can be used for genome editing.

These nucleases sequences and their derived protein allow to efficiently target and/or process double stranded nucleic acid sequences.

Their derived proteins are specific chimeric protein monomers composed of a core scaffold comprising Repeat Variable Dipeptide regions (RVDs) having binding specificity to a target sequence, to which is fused a catalytic domain to its N-terminal portion. This catalytic domain, which can be a monomer of a nuclease, is placed at a position to possibly interact with another catalytic domain fused to another TAL monomer, such that, when both monomers are binding to their respective target DNA sequence, both catalytic domains will form catalytic entity likely to process DNA in the proximity of this target sequence.

The international patent application WO2011/072246 describes a method for modifying the genetic material of a cell with the use of this Transcription Activator-Like Effector nuclease (also called DNA enzyme) nucleotide sequence or its derived protein, wherein the TAL Effector Nuclease will bind to and will process the target DNA.

This method will comprise also the step of providing to the cell, a nucleic acid sequence comprising a sequence being homologous to at least a portion of the target DNA sequence, such that a homologous recombination could occur between the target sequence portion(s) and its corresponding nucleic acid portion(s) disposed between the sequence of the gene of interest.

A transformation of a cell could be obtained either by using one or more vectors comprising the sequences encoding the TALEN protein or by introducing directly the corresponding derived protein into the cell through a mechanic injection, through the means of a bacterial secretion system or through electroporation.

This method has been already used for the transformation of various cells through the use of Transcription Activator-Like proteins, especially type II restriction endonuclease (such as FokI).

However, in the field of plants genetics, transformation methods are complicate, not always efficient, and time consuming. This is particularly true for several plant species, such as sugar beet cells and plants which are reluctant to genetic transformation.

Therefore, it exists a clear need for the improvement of genetic methods dedicated to transformation especially gene targeting of sugar beet plants.

Therefore, the present invention aims to provide a new method and tools for gene targeting of sugar beet cells and plants which allow its genetic transformation, especially genetic targeting and editing of sugar beet protoplasts.

### SUMMARY OF THE INVENTION

The present invention is related to the transformation method of sugar beet protoplasts comprising the steps of:
obtaining protoplasts from stomate guard cells isolated from a sugar beet (Beta vulgaris) plant,
transforming these protoplasts with a nucleic acid construct comprising a nucleotide sequence of interest and Transcription Activator-Like Effector Nucleases (TALEN) sequences or one or more vectors comprising sequences encoding these Transcription Activator-Like Effector Nucleases (TALEN) sequences, wherein these Transcription Activator-Like Effector Nucleases targeting and processing a target (DNA) sequence of the protoplast and replace this target (DNA) sequence through homologous recombination with the nucleic acid construct comprising the nucleotide sequence of interest,
possibly applying to an in vitro culture of these protoplasts, a medium (such as one or more herbicide(s)) that is toxic, preferably lethal to the in vitro culture of the protoplasts, and
regenerating sugar beet plants from the cell culture, preferably from the surviving protoplasts having integrated the nucleic acid construct comprising the sequence of interest that possibly renders the transformed cell resistant to the toxic, preferably lethal activity of the applied medium (such as one or more herbicide(s)).

Preferably, in the method according to the invention, the nucleic acid construct further comprises one or more regulatory sequences for expression of the nucleotide sequence of interest in sugar beet protoplasts, cells, tissues (calli) and/or plants.

Preferably, in the method according to the invention, the applied medium to the in vitro culture of protoplasts comprises one or more ALS inhibitors (herbicide(s)) selected from the group consisting of sulfonylurea herbicides, sulfonylaminocarbonyltrazolinone herbicides, imidazolinone herbicides, triazolopyrimidine herbicides, pyrimidinyl(thio)benzoate herbicides or a mixture thereof.

More preferably, these sulfonylurea herbicides are selected from the group consisting of foramsulfuron, iodosulfuron, amidosulfuron, ethoxysulfuron, chloramsulfuron or a mixture thereof. More preferably, in the method according to the invention the ALS inhibitor(s) (herbice(s)) is (are) applied at a concentration comprised between (about) 5×10⁻⁹M and (about) 1×10⁻⁶M for foramsulfuron, and between (about) 5×10⁻¹¹M and (about) 5×10⁻¹⁰M for ethoxysulfuron.

In the method according to the invention, the sequence of interest encodes a peptide or protein conferring (or involved in) a resistance to one or more herbicide(s), resistance to insects, resistance to nematodes, resistance to plant diseases, resistance to viral infections, resistance to stress (such hydric or saline stress), may encode one or more enzymes and/or may encode a peptide or protein having antibacterial or antifungal properties.

The nucleic acid construct used in the method according to the invention may also use or be present in a vector that comprises one or more regulatory sequence(s) for expression of the nucleic sequence of interest into a sugar beet protoplast, cell, tissue and/or plant. These regulatory sequences are preferably selected from the group consisting of promoter(s) or transcription termination and/or poly-A signal sequence(s), more preferably the CAMV35S promoter sequence and the Nos terminator sequence obtained from agrobacterium tumefaciens.

When the nucleic sequence of interest is the BVALS 113 sequence carrying a sequence mutation at amino acid 113 position from an L-Alanine (Ala) to an L-tyrosine (Tyr) compared to its corresponding wild-type ALS sequence, the preferred and suitable ALS inhibitor(s) (herbicides) is (are) one of the above mentioned herbicides, more preferably foramsulfuron and/or ethoxysulfuron.

Furthermore, the person skilled in the art may select other adequate herbicides which interact with the above mentioned ALS sequence and wherein the above mentioned mutation (at amino acid 113 position from L-Alanine (Ala) to L-tyrosine (Tyr) compared to its wild type corresponding ALS sequence) may render protoplast cell tissue and plant resistance to this ALS inhibitor (herbicide) or mixture of AlS inhibitors (herbicides).

In the method according to the invention, the preferred protoplasts are stomate guard cell protoplasts which have the ability to divide (grow) and to originate viral sugar beet callus when grown in suitable culture media. A callus refers to a mass of undifferentiated cells which can be obtained from explants, such as embryos or parenchyma-derived explants from leaves or a cotyledon. According to the invention, the callus is preferably the result of the growth of well-regenerating (stomatate) guard cell protoplasts, having the capacity to develop shoots or to regenerate into viable sugar beet plants when grown in appropriate culture media, such as polymer-containing medium (preferably an alginate or an agarose containing medium).

The present invention also relates to the protoplast(s), cell(s), tissue(s) (calli) or plant(s) obtained by the transformation method according to the invention, more particularly to a sugar beet plant integrating in its genome the nucleotide sequence of interest, preferably one or more of the sequences above described.

The present invention will now be described in detail in the following detailed description of the invention in reference to the enclosed figures presented as non-limited embodiment of the present invention.

### SHORT DESCRIPTION OF THE DRAWINGS

FIG. 1 represents the introduction of ALS113 mutation in sugar beet protoplasts using specific TALEN sequence and donor matrix containing the mutation along with the flanking sequences for recombination according to the invention.

FIG. 2 represents the T7E1 assay for sugar beet BvALS TALEN activity validation according to the invention.

FIG. 3 represents two plant expression vectors with specific Transcription Activator-Like Effector Nucleases (TALEN) sequence expressed in protoplasts.

FIGS. 4 to 6 represent the sequences of donor matrix sequences according to the invention.

### DETAILED DESCRIPTION OF THE INVENTION

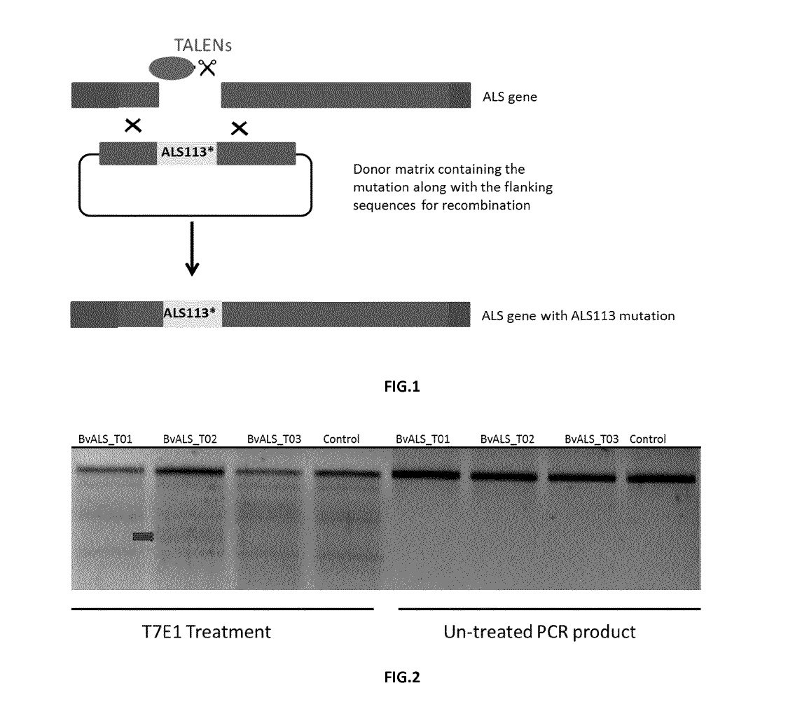

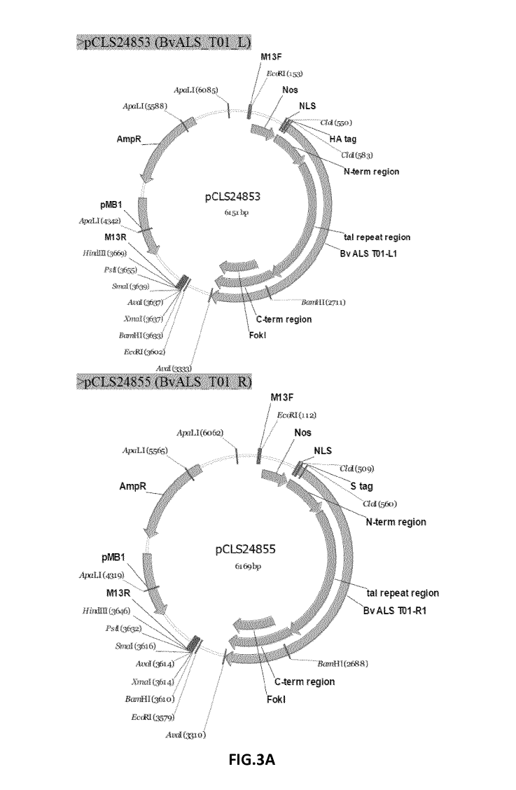

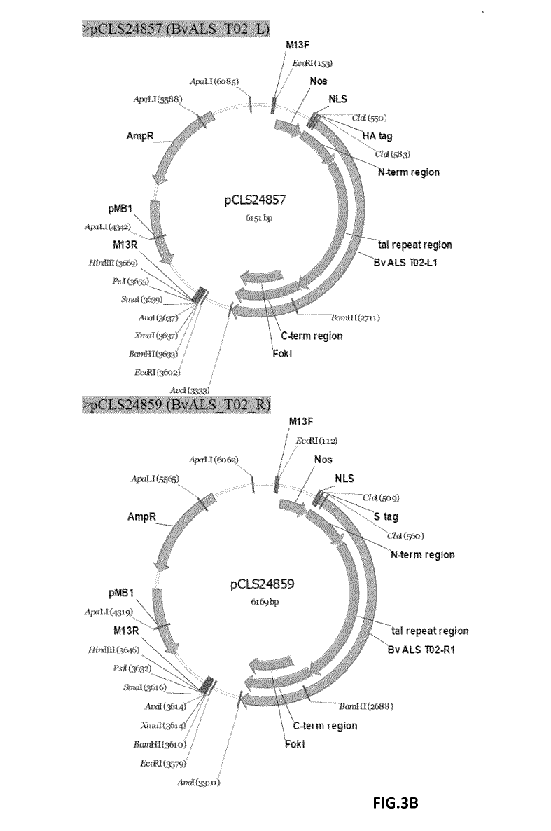

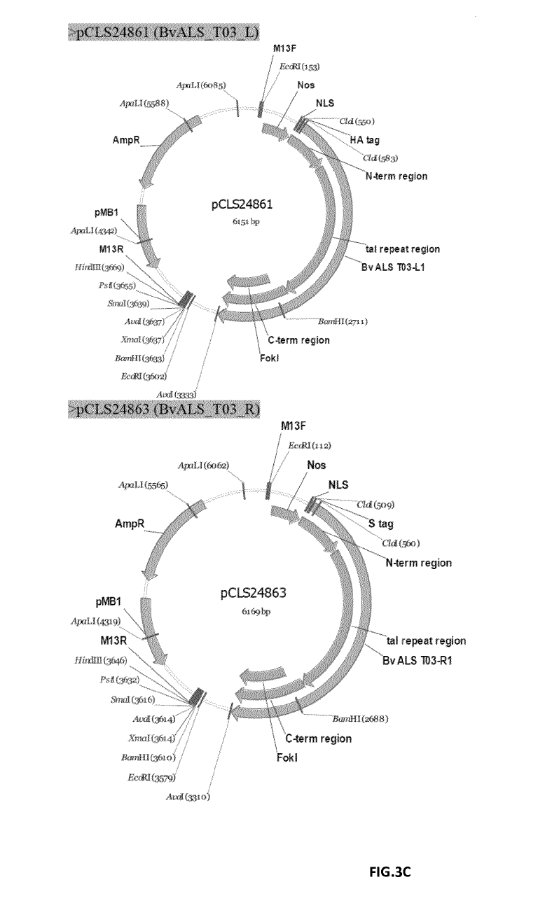

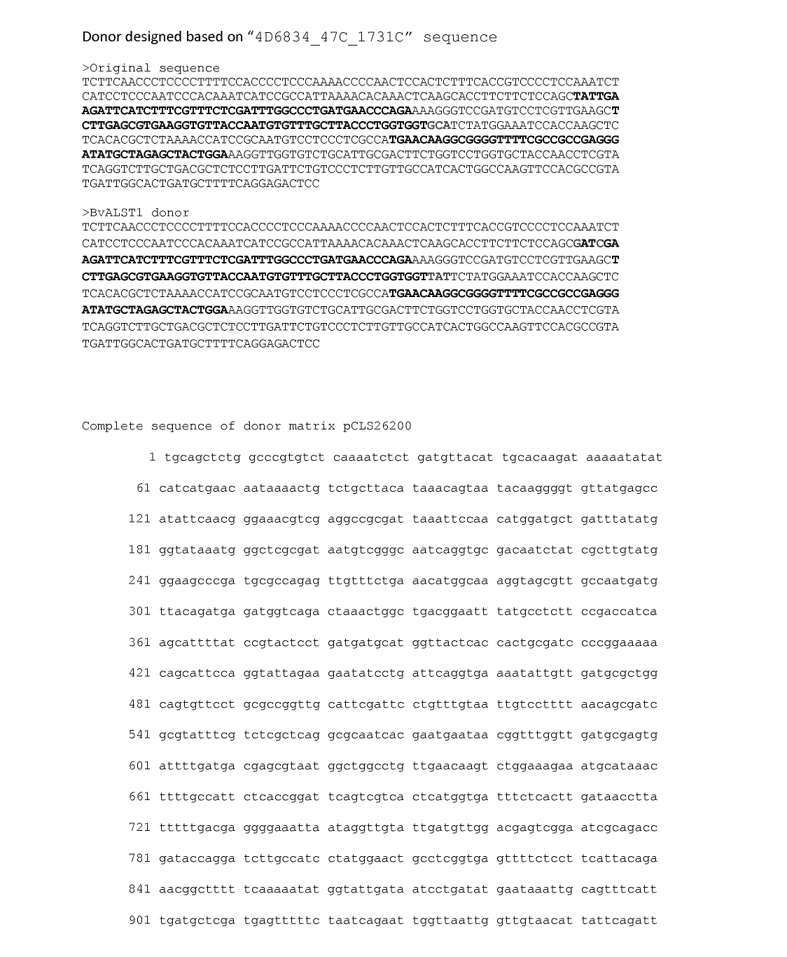

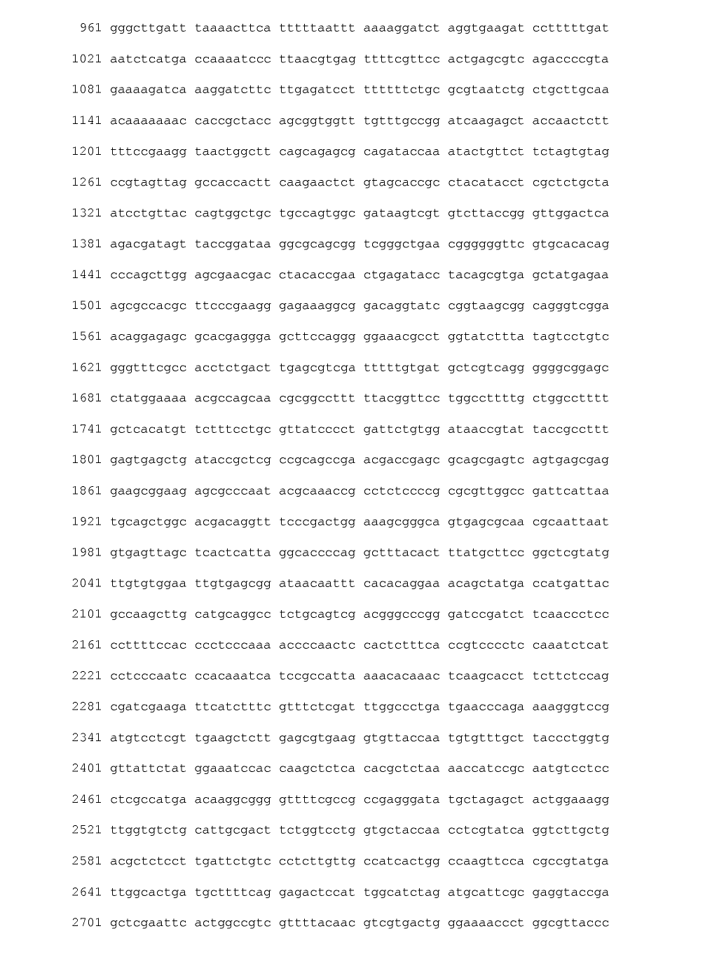

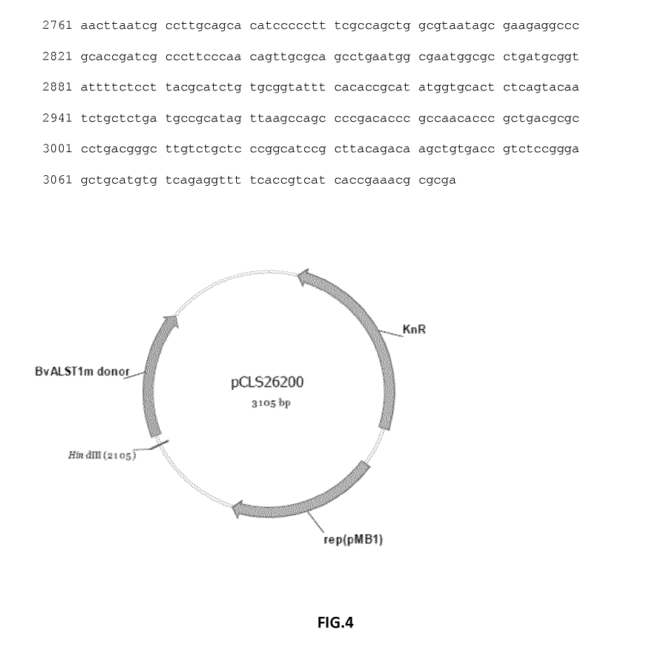

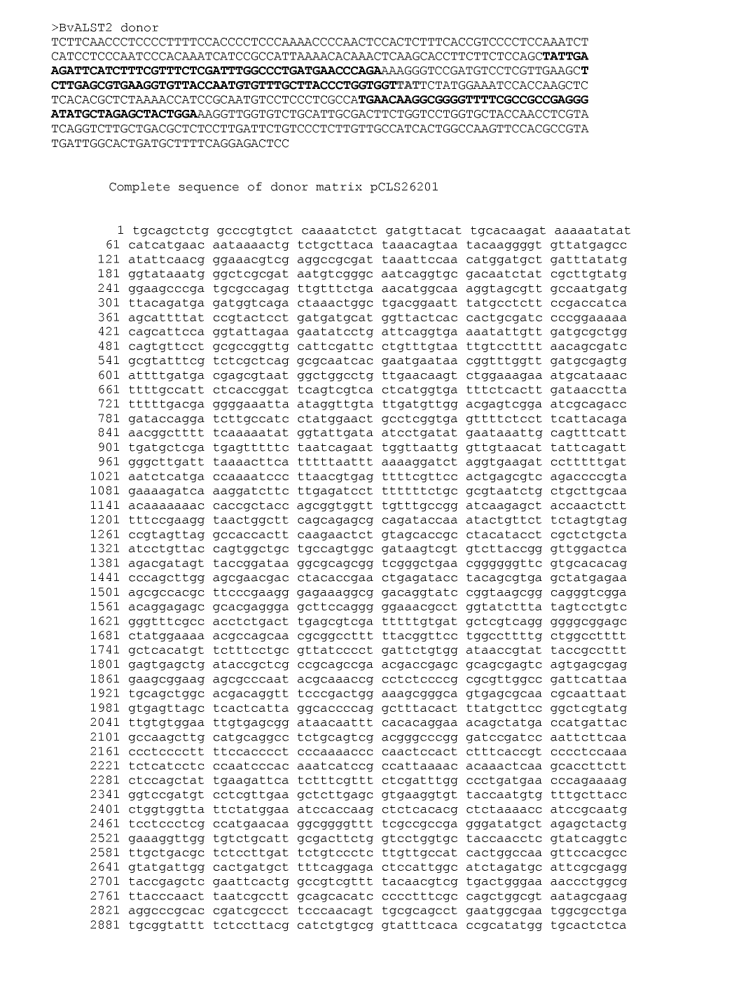

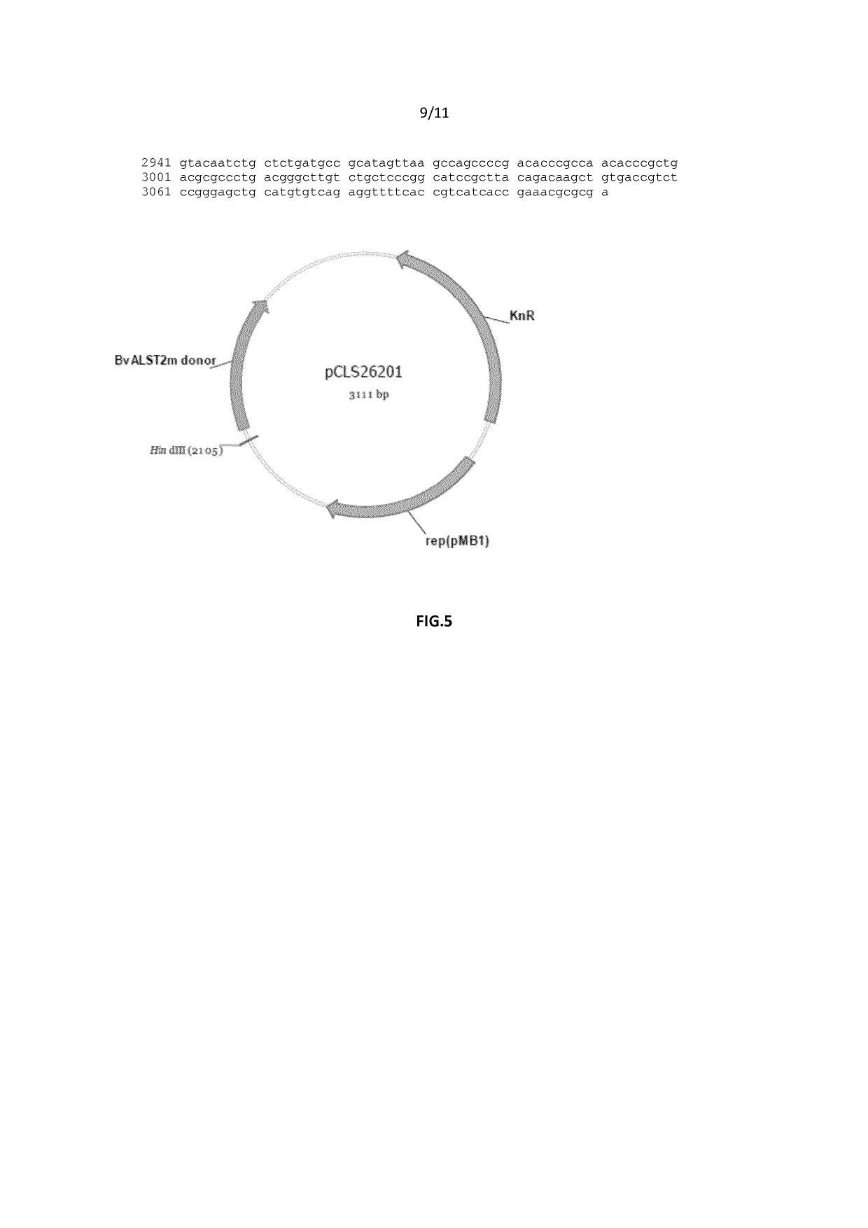

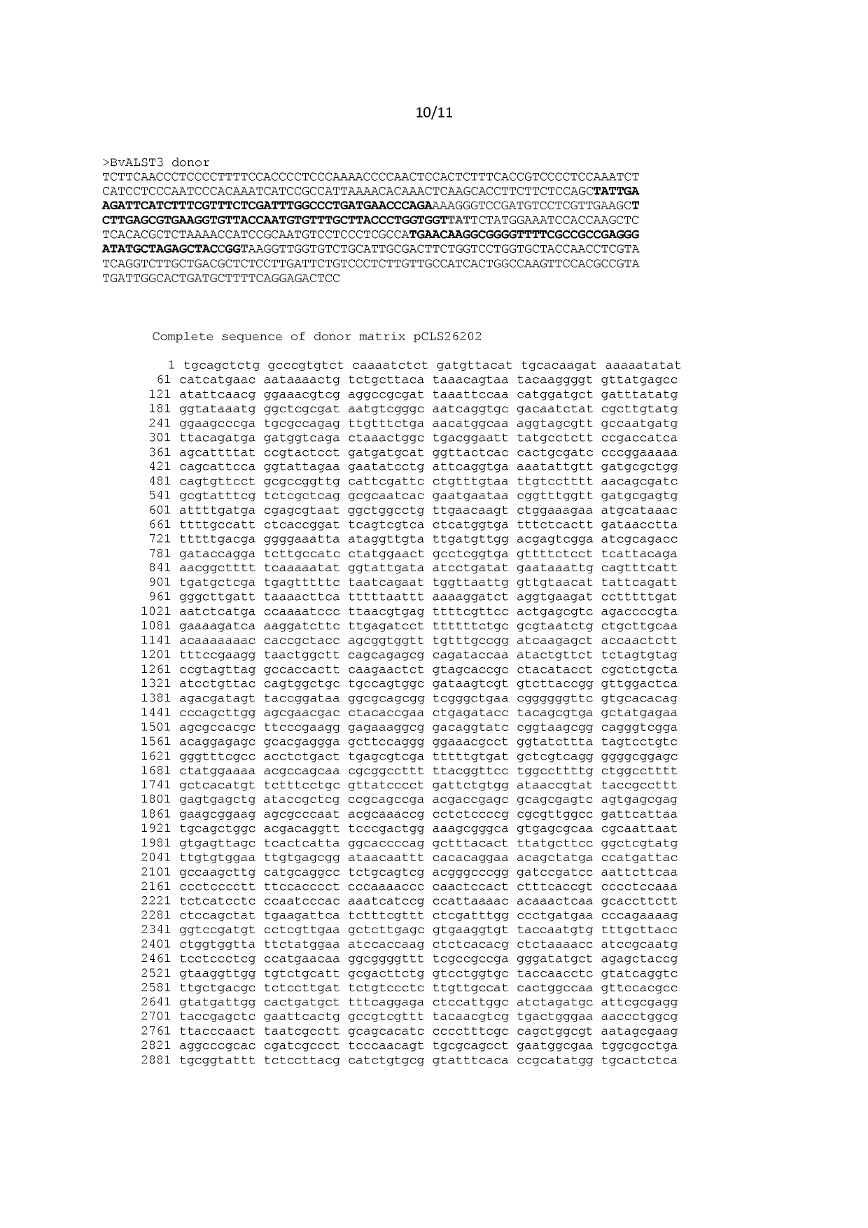

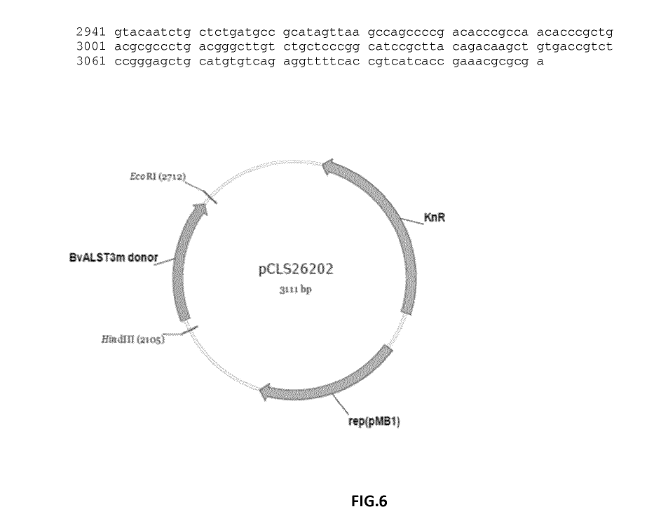

The introduction of specific mutation in sugar beet ALS gene for providing resistance against ALS-inhibitors using TALEN technology is illustrated in the FIG. 1.

CPS developed TALENs (TALEN sequences) based on their yeast screening platform and provided SV with 3 engineered TALEN couples directed against 3 different target sites in the ALS sequence (target sites in close proximity to ALS113 mutation site). All three TALEN pairs came in plant expression vectors along with the three specific donor matrix for each TALEN. When both the TALEN pair and the donor matrix were co-transformed together, the TALEN pair making a double stranded cleavage at specific site and then the donor matrix contacting the specific mutation (ALS113) in inserted into the cleavage site based on the homologous recombination of flanking sequences.

Three TALENS (TALEN sequences) were designed and produced for introducing a point mutation (A113Y, GCA to TAT) into Sugar Beet 3′ ALS gene. Two ALS homologous loci were identified from Sugar Beet (Beta vulgaris) genome and either of them can be used as the target site.

TABLE 1
List of TALEN produced and validated by yeast assay

| TALEN pairs | Plasmid name | gal37 (sd) | gal30 (sd) |
|---|---|---|---|
| BvALS_T01.1 | pCLS24852-pCLS24854 | 0.85 (0.02) | 0.87 (0.06) |
| BvALS_T02.1 | pCLS24856-pCLS24858 | 0.83 (0.03) | 0.84 (0.06) |
| BvALS_T03.1 | pCLS24860-pCLS24862 | 0.91 (0.03) | 0.90 (0.06) |

Three TALEN pairs were synthesized for making the double strand break in the ALS gene near the mutation site. The objective was to measure the efficiency of these three TALEN's by using deep sequencing method to look for the NHEJ (Non-Homologous End Joining) activities and select the one with higher efficiency for carrying sugar beet transformation along with the donor matrix.

TALEN sequences:
BvALS_T01:
TATTGAAGATTCATCTTTCGTTTCTCGATTTGGCCCTGATGAACCCAGA
BvALS_T02:
TCTTGAGCGTGAAGGTGTTACCAATGTGTTTGCTTACCCTGGTGGTGCA
BvALS_T03:
TGAACAAGGCGGGGTTTTCGCCGCCGAGGGATATGCTAGAGCTACTGGA

The efficiency in cutting the double stranded DNA for each TALEN pair was assessed by Sugar Beet protoplast transformation using standard direct gene transformation protocol. After the transformation process, the protoplast were incubated for 24 hours at a temperature of about 26° C. After the incubation, the resulting protoplast were frozen and the DNA isolation was performed according to the same standard protocol. The sequences flanking TALENs Recognition Sites were amplified by PCR. Purified PCR products were then sent for 454 deep sequencing. The sequences were analyzed by bioinformatics to identify the presence of targeted deletions or insertions resulting from NHEJ (Non-Homologous End Joining) events.

Based on the limited 454 sequencing results, TALEN BvALS_T03 cleavage activity (˜2.2% NHEJ mutagenesis rate, see Table 2) were identified. Another approach using the cloning of the PCR fragments and run SANGER sequencing which is semi-quantitive, was used to identify that both BvALS_T02 and BvALS_T03 had good cleavage activities (˜5%, Table 2)

TABLE 2
Sequencing results of PCR products amplified from
TALEN transformed sugar beet protoplast DNA.

| | | # 454 | # seq with | % of | Sanger | # seq | |
|---|---|---|---|---|---|---|---|
| Sample | TALEN | sequence | In/Del | In/Del | seq | with Del | % of Del |
| 1 | BvALS_T01 | 190 | 1 | 0.53 | 94 | 0 | 0 |
| 2 | BvALS_T02 | 123 | 0 | 0 | 94 | 4 | 4.26 |
| 3 | BvALS_T03 | 2883 | 63 | 2.19 | 94 | 6 | 6.38 |
| 4 | N/A | 3244 | 0 | 0 | n/a | n/a | n/a |

T7E1 is an endonuclease that recognizes mismatched double strand DNA and makes cleavage on the mismatched sites. When the PCR products (amplified sequences) were denatured and re-annealed, single strand DNA (sequences) with and without deletions could make double strand DNA (sequence) that has mismatches. Those DNA (sequences) were target for T7E1. Compare to 454 sequencing assay, T7E1 assay was simple and not expensive, but the skilled person can detect TALEN activities that exceed 5%.

FIG. 2 shows the T7E1 assay results. Since there were SNPs between the two ALS genes in sugar beet genome, T7E1 assay indicated some default cuttings even in wild-type material. Those banding pattern may cover the cuttings from BvALS_T01 and BvALS_T03 cleavages. The extra was cutting from BvALS_T02 samples (pointed in FIG. 2), indicating BvALS_T02 had significant cutting activity.

Based on the results from different validation analysis, both BvALS_T02 and BvALS_T03 were good TALEN sequences. Given the fact that the donor for BvALS_T02 did not contain any extra mutation other than the three planned mutation, the BvALS_T02 and its donor were selected for transformation in sugar beet to introduce ALS113 mutation in sugar beet ALS gene.

Sugar beet transformation with TALEN and donor matrix combination

The inventors had initiated the transformation experiments with TALEN EvALS_T02 (pCLS24856-pCLS24858) and its corresponding donor matrix BvALST2 (pCLS26201) to test the efficiency of targeted insertion carrying the mutated ALS. Once the efficiency was determined, the transformation experiments were initiated to regenerate the plants carrying the mutation using the killing curve concentration determined for the selected ALS inhibitor herbicide(s), preferably foramsulfuron and ethoxysulfuron.

1.-9. (canceled)

10. A method for gene editing of sugar beets comprising the steps of:
obtaining protoplasts from stomate guard cells from a sugar beet plant;
introducing in to said protoplasts an engineered nuclease to bind and cut a specific DNA sequence in the genome of said protoplast, wherein said nuclease targets and processes a gene of said protoplast; and
regenerating sugar beet plants from said protoplasts culture.

11. The method according to claim 10, wherein the engineered nuclease is a Transcription Activator-Like Effector Nucleases (TALEN).

12. The method according to claim 10, further comprising the step of introducing into the protoplasts a nucleic acid molecule for editing the targeted gene, said nucleic acid molecule being a donor matrix containing the mutation to introduce along with flanking sequences for recombination.

13. The method according to claim 12 further comprising the step of applying to an in vitro culture of the protoplasts a medium that is toxic to the in vitro culture of the protoplasts, but that is not toxic for the protoplasts having a correctly edited gene.

14. The method according to claim 13, wherein the nucleic acid molecule for editing the targeted gene comprises a fragment of SEQ ID NO:11 so as to elicit an expression of SEQ ID NO:12 by the protoplasts, and wherein the medium that is toxic for the in vitro culture of protoplasts comprises one or more ALS inhibitors.

15. The method according to claim 14, wherein the one or more ALS inhibitor(s) is selected from the group consisting of sulfonylurea herbicides, sulfonylaminocarbonyltrazolinone herbicides, imidazolinone herbicides, triazolopyrimidine herbicides, pyrimidinyl(thio)benzoate herbicides or a mixture thereof.

16. The method according to claim 15, wherein the sulfonylurea herbicides are selected from the group consisting of foramsulfuron, iodosulfuron, amidosulfuron, ethoxysulfuron, chloramsulfuron or a mixture thereof.

17. The method according to the claim 16, wherein the ALS inhibitor is applied at a concentration between 5×10−9M and 1×10−6M for foramsulfuron, and between 5×10−11M and 5×10−10M for ethoxysulfuron.

18. The method according to claim 10, wherein the sequence of interest encodes a peptide or protein selected from the group consisting of peptides or proteins conferring resistance to one or more herbicides, resistance to insects, resistance to nematodes, resistance to plant diseases, resistance to viral infections, resistance to stress, encoding an enzymatic activity and having antibacterial or antifungal properties.

19. The method according to claim 10, wherein the nucleotide sequence of interest is introduced in the form of a nucleic acid construct further comprising one or more regulatory sequences allowing expression of said nucleotide sequence of interest into sugar beet plant protoplasts.

20. The method of claim 18, wherein the one or more regulatory sequences are selected from the group consisting of promoter transcription termination sequence(s) and poly-A signal sequence(s).

21. The method of claim 10, wherein the engineered nuclease is introduced in the form of a nucleic acid molecule.

22. The method of claim 21, wherein the nucleic acid molecule is a vector.

23. The method of claim 10, wherein the engineered nuclease is introduced in the form of a protein.

24. The method of claim 10, further comprising the step of allowing homologous recombination with an exogenous nucleic acid construct comprising a nucleotide sequence of interest.

25. A sugar beet cell expressing SEQ ID NO:12, wherein said SEQ ID NO:12 is produced by an endogenous ALS gene after edition.


SEQUENCE LISTING (12 sequences)

SEQ ID NO: 1 - DNA, 49 bp, Artificial
  TALEN sequence
tattgaagattcatctttcgtttctcgatttggccctgatgaacccaga

SEQ ID NO: 2 - DNA, 49 bp, Artificial
  TALEN sequence
tcttgagcgtgaaggtgttaccaatgtgtttgcttaccctggtggtgca

SEQ ID NO: 3 - DNA, 49 bp, Artificial
  TALEN sequence
tgaacaaggcggggttttcgccgccgagggatatgctagagctactgga

SEQ ID NO: 4 - DNA, 521 bp, Artificial
  Original sequence of donor based on 4D6834_47C_1731C
tcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactcc

SEQ ID NO: 5 - DNA, 521 bp, Artificial
  BVALST1 donor based on 4D6834_47C_1731C
tcttcaaccctccccttttccacccctcccaaaaccccaactccactctttcaccgtcccctccaaatctcatcctcccaatcccacaaatcatccgccattaaaacacaaactcaagcaccttcttctccagcgatcgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactcc

SEQ ID NO: 6 - DNA, 3105 bp, Artificial
  complete sequence of donor matrix pCLS26200
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgatcttcaaccctccccttttccacccctcccaaaaccccaactccactctttcaccgtcccctccaaatctcatcctcccaatcccacaaatcatccgccattaaaacacaaactcaagcaccttcttctccagcgatcgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccattggcatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 7 - DNA, 521 bp, Artificial
  BvALST2 donor
tcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactcc

SEQ ID NO: 8 - DNA, 3111 bp, Artificial
  donor matrix pCLS26201
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgatccaattcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccattggcatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 9 - DNA, 521 bp, Artificial
  BvALST3 donor
tcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctaccggtaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactcc

SEQ ID NO: 10 - DNA, 3111 bp, Artificial
  Complete sequence of donor matrix pCLS26202
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgatccaattcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctaccggtaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccattggcatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 11 - DNA, 1998 bp, Beta vulgaris
  CDS (1)..(1998): BVALS A113Y
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 12 - protein, 665 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGYSMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY